(19) United States
(12) Patent Application Publication
(10) Pub. No.: US 2006/0029503 A1
(43) Pub. Date: Feb. 9, 2006

(54) Plunger pump and method of controlling discharge of the pump

(76) Inventors: Norio Takehana, Iwate (JP); Hideaki Kusanagi, Iwate (JP)

Correspondence Address: WENDEROTH, LIND & PONACK, L.L.P., 2033 K STREET N. W., SUITE 800, WASHINGTON, DC, 20006-1021, US

(21) Appl. No.: 11/194,602

(22) Filed: Aug. 2, 2005

(30) Foreign Application Priority Data
Aug. 4, 2004 (JP) 2004-227544

(51) Int. Cl. A61M 1/00; F04B 41/06

(52) U.S. Cl. 417/437; 417/521

(57) ABSTRACT

There are provided a plunger pump and method of controlling discharge of the pump capable of obtaining a desired compression rate, changing a discharge amount with ease without changing a stroke of the plunger and/or the inner diameter of a sliding hole of the plunger, and discharging a constant amount of fluid with accuracy while preventing the fluid from leaking. A plunger pump 1 has a cylinder 2 having an inlet 8 to suck a fluid and an outlet 10 to discharge the sucked fluid, a continuous hole 6 which is formed inside the cylinder 2 and communicated with the outlet 10, a plunger 4 which is inserted in the continuous hole 6 to be slidable and forms a pump chamber 20 to suck and discharge the fluid with the outlet 10 where the pump chamber 20 is formed between the plunger 4 and the outlet 10, and a fluid suction passage which is formed in the cylinder or the plunger, to suck a fluid into the pump chamber, wherein an opening 19 of the fluid suction passage 12 opened to the pump chamber 20 is opened and closed by the plunger 4 sliding inside the continuous hole 6.

### BACKGROUND OF THE INVENTION

1. Field of the Invention

The present invention relates to a plunger pump that sucks a constant amount of fluid from a fluid source to discharge and to a method of controlling discharge of the pump.

2. Description of Related Art

In plunger pumps that suck and discharge a fluid by reciprocating motion of a plunger, various manners have conventionally been proposed for the driving form of the plunger, arrangement form of valves and the like. For example, Patent Documents 1 to 3 each disclose a plunger type electromagnetic pump that reciprocates a plunger with electromagnetic activation force generated by applying current to an electromagnetic coil.

FIG. 8 illustrates a plunger type electromagnetic pump of the same type as that of the electromagnetic pump disclosed in Patent Documents 1 to 3. As shown in the figure, a plunger pump 100 has a cylinder 102, and a plunger 104 that is inserted in the cylinder 102 to be slidable. More specifically, the cylinder 102 has a suction side S and discharge side D, and the plunger 104 is inserted slidably in a continuous hole 106 formed on the suction side S of the cylinder 102. The plunger 104 is provided with an inlet 108 to suck a fluid such as lubricating oil from a fluid source such as a reservoir tank not shown, and the cylinder 102 is provided at its discharge side D with an outlet 110 that discharges the fluid sucked through the inlet 108 and that is communicated with the continuous hole 106.

The plunger 104 is slid inside the continuous hole 106 by electromagnetic activation force generated by applying current to an electromagnetic coil of a solenoid not shown, and a pump chamber 120 to suck and discharge the fluid is formed between the plunger 104 and outlet 110.

Further, a fluid suction passage 112 that connects the pump chamber 120 and inlet 108 is formed in the plunger 104 along the axis of the plunger 104. In this case, an opening 112a of the fluid suction passage 112 opened to the pump chamber 120 is opened and closed by a suction valve 125 with sliding of the plunger 104. The suction valve 125 is provided inside the pump chamber 120, and comprised of a sphere-shaped valve body 125a, and a spring 125b that is inserted between the valve body 125a and outlet 110 and that supports the valve body 125a for the cylinder 102.

Furthermore, the outlet 110 is provided with a discharge valve 130 that constitutes a one-way valve. The discharge valve 130 is comprised of a sphere-shaped valve body 130a and compression spring 130b, usually presses the valve body 130a against a base 110a of the outlet 110 by force of the compression spring 130b to close the outlet 110, and only when a pressure exceeding the force of the compression spring 130b is generated inside the pump chamber 120, opens the outlet 110.

In the plunger pump 100 configured as described above, when the current is not applied to the electromagnetic coil (OFF state), since a driving core of the solenoid not shown escapes and the plunger 104 is pulled back to the bottom dead center of its stroke, the valve body 125a of the suction valve 125 gets away from the opening 112a of the fluid suction passage 112, the opening 112a is opened, and the pump chamber 120 is communicated with the inlet 108. Accordingly, the fluid from the fluid source flows into the pump chamber 120.

Subsequently, when the current is applied to the electromagnetic coil (ON state) at predetermined timing, the driving core of the solenoid goes forward to push the plunger 104 in the continuous hole 106, and the valve body 125a of the suction valve 125 comes into contact with the opening 112a of the fluid suction passage 112 to close the opening 112a. Then, when the plunger 104 is further pushed in the hole 106 against the force of the spring 125b, the pressure inside the pump chamber 120 increases with the close state of the opening 112a kept by the spring 125b. When the pressure exceeds the force of the spring 130b of the discharge valve 130, the valve body 130a gets away from the base 110a, the outlet 110 is opened, and the fluid in the pump chamber 120 is discharged from the outlet 110. In addition, the fluid discharged from the outlet 110 is guided to a lubricant target portion of an operating body such as an engine via a pipe-shaped connection cap 135 provided on the discharge side D of the cylinder 102.

Further, when the plunger 104 is pushed in the continuous hole 106 as described above and reaches the top dead center of its stroke, at this point the application of current to the electromagnetic coil is halted (OFF state), and the plunger 104 is pulled back again to the bottom dead center of its stroke again by the escape operation of the driving core of the solenoid. Then, when the plunger 104 gets away from the valve body 125a of the suction valve 125 to open the opening 112a of the fluid suction passage 112, the pump chamber 120 and inlet 108 are communicated, thereby shifting to the suction operation as described previously.

In addition, a series of suction/discharge operation as described above is carried out repeatedly with ON/OFF of the application of current to the electromagnetic coil as one cycle, as shown in FIG. 8(d)
[Patent Document 1] Patent No. 3345332
[Patent Document 2] Patent No. 3429719
[Patent Document 3] JP H08-270571

In the conventional pump structure as shown in FIG. 8, two-component valves (suction valve 125 and discharge valve 130) each comprised of the sphere-shaped valve body and spring are provided on both the suction side S and discharge side D. There arise problems that the number of components is large, the valve structure is complicated, the assembly is also complicated, and that the entire plunger pump is upsized. Such upsizing is further promoted by serially arranging the suction valve 125 and discharge valve 130 along the fluid suction/discharge direction.

Further, in the conventional pump structure as shown in FIG. 8, since the suction valve 125 is disposed inside the pump chamber 120 constituting a pump chamber, a dead volume increases in the pump chamber 120, and it is not possible to effectively use the entire inner capacity of the pump chamber 120 as a pump chamber. Therefore, the compression rate decreases, and substantially adverse effects are exerted in increases in discharge pressure and in air exclusion.

Furthermore, in the conventional pump structure as shown in FIG. 8, since the opening 112a is opened and closed by the plunger 104 repeatedly contacting the valve body 125a of the suction valve 125 (plunger 104 tapping the valve body 125a), as well as noise and vibration occurring, there is a fear that the seal characteristic of the pump chamber 120 deteriorates due to wear of the suction valve 125 and the like.

Moreover, in the conventional pump structure as shown in FIG. 8, changing a discharge flow amount requires changes in stroke of the plunger 104 and in inner diameter of the continuous hole 106, but changing the inner diameter of the continuous hole 106 results in complicated processing.

A series of problems as described above becomes more pronounced in multi-discharge type plunger pump comprised of a plurality of pump structures as shown in FIG. 8 to discharge the fluid concurrently from a plurality of outlets. Particularly, in such a multi-discharge type plunger pump, in the case of operating a plurality of plungers 104 at the same time by a common driving part and supplying different flow amounts from the outlets 110 at a constant discharge pitch with the same stroke set on all the plungers 104, as described previously, it is necessary to change an inner diameter of the continuous hole 106 for each of the plungers 104. In such a case, the assembly process becomes complicated, as well as the processing process becoming complicated, and further, the management process may increase.

In the conventional pump structure as shown in FIG. 8, in order to reduce power consumption, the time of applying the current to the electromagnetic coil (duration time of ON state) is fixed at a required minimum time, and the time of halting the application of current to the electromagnetic coil (duration time of OFF state) is adjusted to determine the frequency of driving the solenoid (see FIG. 8(d)). Therefore, the current OFF time is inevitably long that is the fluid suction step time. However, when the current OFF time thus is long, one problem arises. That is, at the time of fluid suction step (current OFF state), since only the discharge valve 130 closes the flow passage of the fluid in the pump, when the pressure on the discharge side D becomes smaller than the pressure on the suction side S, the pressure to open the discharge valve 130 decreases, and there is a fear of occurrence of minute leakage of the fluid in the base 110a (so-called blow-by phenomenon where the fluid from a fluid source is sucked from the inlet 108 and leaks from the outlet 110 due to the discharge valve 130 and suction valve 125 being both opened). Therefore, when the fluid suction step time (current OFF time) becomes long, the risk of occurrence of minute leakage is increased, it becomes difficult to supply a constant amount of fluid reliably, and the fluid is wasted. In addition, in the conventional pump structure as shown in FIG. 8, even during the discharge process, since there is a possibility that the suction valve 125 is opened by a pressure difference between the suction side S and discharge side D, the blow-by phenomenon may occur.

### SUMMARY OF THE INVENTION

In view of the foregoing, it is an object of the present invention to provide a plunger pump and method of controlling discharge of the pump capable of obtaining a desired compression rate, changing a discharge amount with ease without changing a stroke of the plunger and/or the inner diameter of a sliding hole of the plunger, and discharging a constant amount of fluid with accuracy while preventing the fluid from leaking. Further, it is another object to provide a plunger pump and method of controlling discharge of the pump enabling simplified assembly and miniaturization while preventing expensive oil and fuel from being wasted.

In order to achieve the objects, a plunger pump according to a first aspect of the invention has a cylinder having an inlet to suck a fluid from a fluid source and an outlet to discharge the sucked fluid, a continuous hole which is formed inside the cylinder and communicated with the outlet, a plunger which is inserted in the continuous hole to be slidable and forms a pump chamber to suck and discharge the fluid with the outlet where the pump chamber is formed between the plunger and the outlet, and a fluid suction passage which is formed in the cylinder or the plunger, to suck a fluid into the pump chamber, where an opening of the fluid suction passage opened to the pump chamber is opened and closed by the plunger sliding inside the continuous hole.

According to the plunger pump of the first aspect, since the opening of the fluid suction passage opened to the pump chamber is opened and closed by the plunger itself, in other words, the plunger is provided with the valve function (the plunger serves as a suction valve on the fluid suction side), the need is eliminated of providing a dedicated valve (for example, the suction valve 125 as shown in FIG. 8) on the fluid suction side. Therefore, as compared with the conventional case, the number of components is decreased, the valve structure is simplified, the assembly is also simplified, and it is possible to miniaturize the entire plunger pump.

Further, since any members are not present (such as, for example, the valve body 125a of the suction valve 125 as shown in FIG. 8) that collide with the plunger with opening and closing of the opening of the fluid suction passage, it is possible to minimize the vibration and noise at the time the pump is operating, and to avoid wear of the valve structure and occurrence of failure in sealing inside the pump chamber due to the wear.

In addition, the size and number of the fluid suction passages may be set optionally corresponding to a required discharge amount. Further, as a driving mechanism of the plunger, any of conventionally known manners can be used such as a solenoid, motor, and cam driven by an engine. Furthermore, the fluid suction passage may be formed along the inner surface of the continuous and extend in parallel with the axis direction of the continuous hole, extend in the direction perpendicular to the axis direction of the continuous hole, or extend obliquely to the axis direction of the continuous hole.

A plunger pump according to a second aspect has a valve body that is provided to close the outlet and that opens the outlet only when receiving a predetermined pressure generated inside the pump chamber by the plunger sliding toward the outlet. By this means, in the plunger pump, since a valve member is not needed inside the pump chamber constituting a pump chamber, the issue of the dead volume in the pump chamber is also resolved, and it is possible to use the entire inner capacity of the pump chamber effectively as a pump chamber. The compression rate (pump performance) is thus improved, and it is possible to achieve a desired structure in increases in discharge pressure and air exclusion.

In a plunger pump according to a third aspect in the plunger pump of the first aspect, a discharge amount of the fluid discharged from the outlet is determined by a top dead center of a stroke of the plunger and a position of the opening of the fluid suction passage.

According to the plunger pump of the third aspect, as well as obtaining the same effects and advantages as in the plunger pump of the first aspect, since a discharge amount of the fluid discharged from the outlet is specified by a top dead center of a stoke of the plunger and a position of the opening of the fluid suction passage, it is possible to change a discharge amount (obtain a required discharge flow rate) with ease only by merely changing a position of the opening (accordingly, with simplified processing) without changing (an amount of) the stroke of the plunger and inner diameter of the continuous hole. In other words, only by changing a position of the opening, it is possible to vary a ratio of a suction stroke and discharge stroke to the entire stroke of the plunger. In addition, in the specification, “top dead center” is a position where the plunger is pushed in the continuous hole the deepest.

In a plunger pump according to a fourth aspect in the plunger pump of the first aspect, the fluid suction passage is formed on the inner surface of the continuous hole along the axis direction thereof.

According to the plunger pump of the fourth aspect, as well as obtaining the same effects and advantages as in the plunger pump of the first aspect, since the fluid suction passage is formed on the inner surface of the continuous hole along the axis direction thereof, the extending direction of the fluid suction passage is in accordance with the extending direction of the continuous hole, it is thereby possible to perform processing on the fluid suction passage in the same direction as in processing on the continuous hole, and the processing is thus easy.

In addition, in the above-mentioned constitution, a driving part may be further provided to slide the plunger inside the continuous hole. In this case, the driving part may slide the plunger inside the continuous hole by electromagnetic activation force generated by applying current to the electromagnetic coil. Further, as the fluid, various types of fluids are considered such as oils including lubricant oil and gasoline.

The driving part in a plunger pump of a fifth aspect holds the plunger at the top dead center position of its stroke for a predetermined time.

According to the plunger pump of the fifth aspect, since the plunger is held at the top dead center position of its stroke for a predetermined time, it is possible to minimize the time the plunger is held at the bottom dead center position of its stroke (position having the risk of occurrence of the so-called blow-by phenomenon such that the fluid from a fluid source is sucked from the inlet and leaks from the outlet due to the valve body of the outlet being opened by a pressure difference between the pump suction side and discharge side) in one cycle of suction/discharge operation. In other words, it is possible to reduce to a minimum the time percentage of occurrence of the blow-by phenomenon by a pressure difference between the pump suction side and discharge side, and to secure a proper discharge amount. Particularly, in the case of controlling the flow rate while varying the driving frequency of the pump, the aforementioned advantage is more effective as the driving frequency is lower. As the flow rate is smaller, the adverse effect of minute leakage is more significant, and the blow-by and/or suppression time (OFF time) is longer, whereby the advantage is more useful in discharging a small amount of fluid (a set discharge amount is small in one cycle).

In addition, for a period during which the plunger is held at the top dead center position of its stroke, since the pump chamber is kept at a sealed state (because the opening of the fluid suction passage is closed by the plunger and the inlet and outlet are not communicated with each other basically), the blow-by phenomenon is suppressed. In this case, a clearance seal between the plunger and cylinder largely contributes to suppression of the blow-by phenomenon. In contrast thereto, in the conventional structure as shown in FIG. 8, even if the plunger is held at the top dead center of its stroke, there is a possibility that the suction valve 125 is opened by a pressure difference between the suction side S and discharge side D and/or vibration, and it is thus difficult to completely suppress the blow-by phenomenon.

Further, after the plunger reaches the top dead center position of its stroke, the driving part in a plunger pump of a sixth aspect holds the plunger at the top dead center position for a predetermined time by maintaining a voltage lower than an application voltage to the electromagnetic coil required to slide the plunger to the top dead center position.

According to the plunger pump of the sixth aspect, since the voltage (power to maintain the discharge completion state) applied to the electromagnetic coil to keep the plunger at the top dead center position for a predetermined time is lower than the application voltage (power required to start discharging) required to slide the plunger to the top dead center position, power savings can be achieved (power consumption can be reduced). The thrust in the electromagnetic plunger part becomes the maximum in the discharge completion state, and therefore, the discharge completion state can be maintained sufficiently even when the operation voltage is decreased.

A multi-discharge type plunger pump according to a seventh aspect has a suction duct to suck a fluid from a fluid source, a plurality of cylinder parts each having an inlet communicated with the suction duct and an outlet to discharge the sucked fluid, continuous holes each of which is formed inside respective one of the cylinder parts and communicated with the outlet, plungers each of which is inserted in respective one of the continuous holes to be slidable and forms a pump chamber to suck and discharge the fluid with the outlet where the pump chamber is formed between each of the plungers and the outlet, and a plurality of fluid suction passages each of which is formed in respective one of the cylinder parts or plungers inserted in respective one of the continuous holes to suck a fluid into the pump chamber, where an opening of each of the fluid suction passages opened to the pump chamber is opened and closed by respective one of the plungers sliding in respective one of the continuous holes.

According to the multi-discharge type plunger pump of the seventh aspect, as well as obtaining the same effects and advantages in the first aspect, particularly, even in the case of operating a plurality of plungers at the same time by a common driving part and supplying different flow amounts from the outlets at a constant discharge pitch with the same stroke set on all the plungers, it is only required to change a position of the opening of each of the fluid suction passages without changing an inner diameter of the continuous hole for each of the plungers, and the processing is thus easy. In other words, in a multi-discharge structure that operates a plurality of plungers in conjunction with one another, it is possible to change a setting of flow rate in each pump chamber only by changing a position of the opening of respective one of the fluid suction passages, and variations as a pump can thus be dramatically extended.

Moreover, the invention intends to provide a method of controlling discharge using the plunger pump with each of the above-mentioned structures.

Thus, according to the plunger pump and method of controlling discharge of the pump of the invention, a desired compression rate is obtained, and it is possible to change a discharge amount with ease without changing a stroke of the plunger and/or the inner diameter of a sliding hole of the plunger, and to discharge a constant amount of fluid while preventing the fluid from leaking. Further, it is possible to extremely reduce both the noise and vibration in using the pump. Furthermore, the number of components is decreased, the assembly is simplified, and the size (particularly, longitudinal size) is reduced, thereby enabling miniaturization.

### BRIEF DESCRIPTION OF THE DRAWINGS

FIG. 1(a) is a sectional view of a plunger pump in a suction step according to one embodiment of the present invention;

FIG. 1(b) is a sectional view of the plunger pump in a discharge step according to the one embodiment of the invention;

FIG. 1(c) is a sectional view taken along line B-B of FIG. 1(a);

FIG. 1(d) is a waveform diagram of voltage to apply to an electromagnetic coil with control of suction and discharge;

FIG. 2(a) is a sectional view according to a modification of the plunger pump as shown in FIG. 1;

FIG. 2(b) is a sectional view taken along line C-C of FIG. 2(a);

FIG. 3 is a side view of a multi-discharge type plunger pump according to one embodiment of the invention;

FIG. 4 is a view viewed in the direction of arrow A of FIG. 3;

FIG. 5 is a sectional view of a principal part of the multi-discharge type plunger pump of FIG. 3;

FIG. 6 is a sectional view of a multi-discharge type plunger pump according to another embodiment;

FIG. 7 is a view viewed in the direction of arrow B of FIG. 6;

FIG. 8(a) is a sectional view of a conventional plunger pump in a suction step;

FIG. 8(b) is a sectional view of the conventional plunger pump in a discharge step;

FIG. 8(c) is a sectional view taken along line A-A of FIG. 8(a); and

FIG. 8(d) is a waveform diagram of voltage to apply to an electromagnetic coil with conventional control of suction and discharge.

### DETAILED DESCRIPTION OF THE PREFERRED EMBODIMENTS

Embodiments of the present invention will specifically be described below with reference to accompanying drawings.

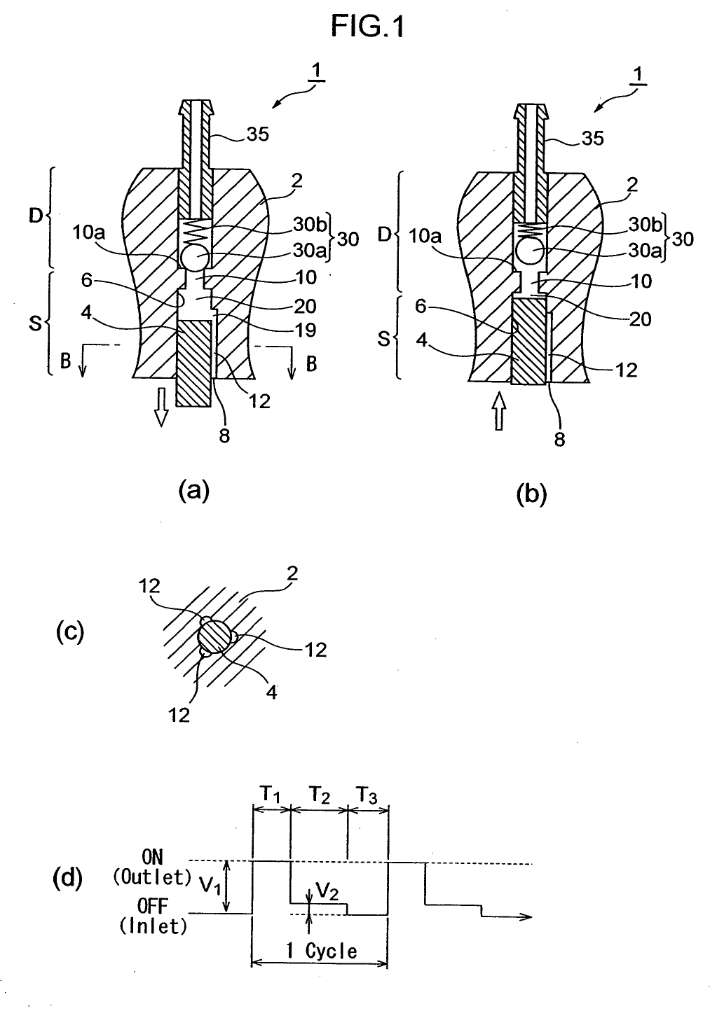

FIG. 1 shows a plunger pump 1 according to one embodiment of the invention. As shown in the figure, the plunger pump 1 of this embodiment is provided with a cylinder 2, and a cylindrical plunger 4 inserted in the cylinder 2 slidably. More specifically, the cylinder 2 has a suction side S and discharge side D, and the plunger 4 is inserted slidably in a circular cross-section continuous hole 6 formed on the suction side S of the cylinder 2. The plunger 4 is provided with an inlet 8 to suck a fluid such as lubricant oil from a fluid source such as a reservoir tank not shown. The cylinder 2 is provided with an outlet 10 that discharges the fluid sucked through the inlet 8 and that is communicated with the continuous hole 6.

The plunger 4 is slid inside the continuous hole 6 by the electromagnetic activation force generated by applying current to an electromagnetic coil of a solenoid (driving part) not shown, and forms a pump chamber 20 to suck and discharge the fluid between the outlet 10 and the plunger 4.

Further, in the cylinder 2 is formed at least one fluid suction passage 12 that connects the pump chamber 20 and inlet 8. Particularly, in this embodiment, three fluid suction passages 12 are formed on the inner surface of the continuous hole 6 along the axis direction thereof at substantially same angle intervals in the circumferential direction of the continuous hole 6 (as the number of the passages, one or more number is applicable). More specifically, each of the fluid suction passages 12 is obtained by cutting part of the inner surface of the continuous hole 6 toward the outside in the diameter direction, and thus formed between the inner surface of the cylinder 2 constituting the continuous hole 6 and the outer surface of the plunger 4, while its one end is terminated at a position spaced a predetermined distance away from the outlet 10 in the axis direction, and the other end is terminated at the end of the continuous hole 6 to form the inlet 8. Accordingly, an opening 19 of the fluid suction passage 12 opened to the pump chamber 20 is opened and closed by the plunger 4 itself sliding inside the continuous hole 6, and an opening degree of the opening 19 of the fluid suction passage 12 to the pump chamber 20 is varied with sliding of the plunger 4.

As described above, the fluid suction passage 12 which connects the pump chamber 20 and the inlet 8 in order to suck a fluid into the pump chamber 20 is formed in the cylinder 2. However, the fluid suction passage 12 can be formed in the plunger 4 to have the same effect.

Further, the outlet 10 is provided with a discharge valve 30 that constitutes a one-way valve. The discharge valve 30 is comprised of a sphere-shaped valve body 30a and compression spring 30b, usually presses the valve body 30a against a base 10a of the outlet 10 by force of the compression spring 30b to close the outlet 10, and only when a pressure exceeding the force of the compression spring 30b is generated inside the pump chamber 20, opens the outlet 10.

The operation of the plunger pump 1 with the above-mentioned structure will be described below together with control of voltage to the electromagnetic coil.

In an OFF state where the current is not applied to the electromagnetic coil (see FIG. 1(d)), a driving core of the solenoid not shown escapes, and the plunger 4 is pulled back to the bottom dead center of its stroke (the position where the plunger 4 is the farthest away from the inlet 10 within a stroke range of the plunger 4). Therefore, as shown in FIG. 1(a), the opening 19 of the fluid suction passage 12 is opened by the plunger 4, and the pump chamber 20 is communicated with the inlet 8. Accordingly, the fluid from the fluid source flows into the pump chamber 20.

Subsequently, when the current is applied to the electromagnetic coil (ON state, see FIG. 1(d)) at predetermined timing, the driving core of the solenoid goes forward to push the plunger 4 in the continuous hole 6, and the opening 19 of the fluid suction passage 12 is closed by the plunger 4. Accordingly, in this state, the pump chamber 20 is kept at a sealed state. When the plunger 4 is further pushed in from this state, the pressure increases inside the pump chamber 20. Then, when the pressure exceeds the force of the spring 30b of the discharge valve 30, the valve body 30a gets away from the base 10a to open the outlet 10, and the fluid inside the pump chamber 20 is discharged from the outlet 10 (a state of FIG. 1(b)). In addition, the fluid discharged from the outlet 10 is guided to a lubricant target portion of an operating body such as an engine via a pipe-shaped connection cap 35 provided on the discharge side D of the cylinder 2.

As described above, the plunger 4 is pushed in the continuous hole 6, and in a stage where the plunger 4 reaches the top dead center of its stroke, all the set discharge amount determined beforehand is discharged from the outlet 10. The set discharge amount in this case is specified by the top dead center of the stroke of the plunger 4 and a position of the opening 19 of the fluid suction passage 12.

Further, when the plunger 4 reaches the top dead center of the stroke, the plunger 4 is held at the top dead center position for a predetermined time. In this case, the voltage to apply to the electromagnetic coil is maintained at a voltage value V₂ lower than a voltage value V₁ required to slide the plunger 4 to the top dead center position (see FIG. 1(d)). In other words, at the time the plunger 4 reaches the top dead center of its stroke, the voltage applied to the electromagnetic coil is decreased from V₁ to V₂, and kept at V₂ for a predetermined time. In the case where the discharge step is performed by electromagnetic force and the suction step is performed by spring force in this constitution, the thrust of the electromagnetic plunger part becomes the maximum in the discharge completion state (where the plunger 4 reaches the top dead center position), and therefore, it is possible to maintain the discharge completion state sufficiently even when the operation voltage is decreased from V₁ to V₂. Herein, time T₂ to hold the plunger 4 at the top dead center position may be, for example, substantially half the time corresponding to one cycle of the suction/discharge operation, and is preferably set such that ON time T₁ plus the top dead center holing time T₂ exceeds OFF time T₃. More specifically, the discharge completion time may be maintained for all the time except the time required for the suction and discharge steps.

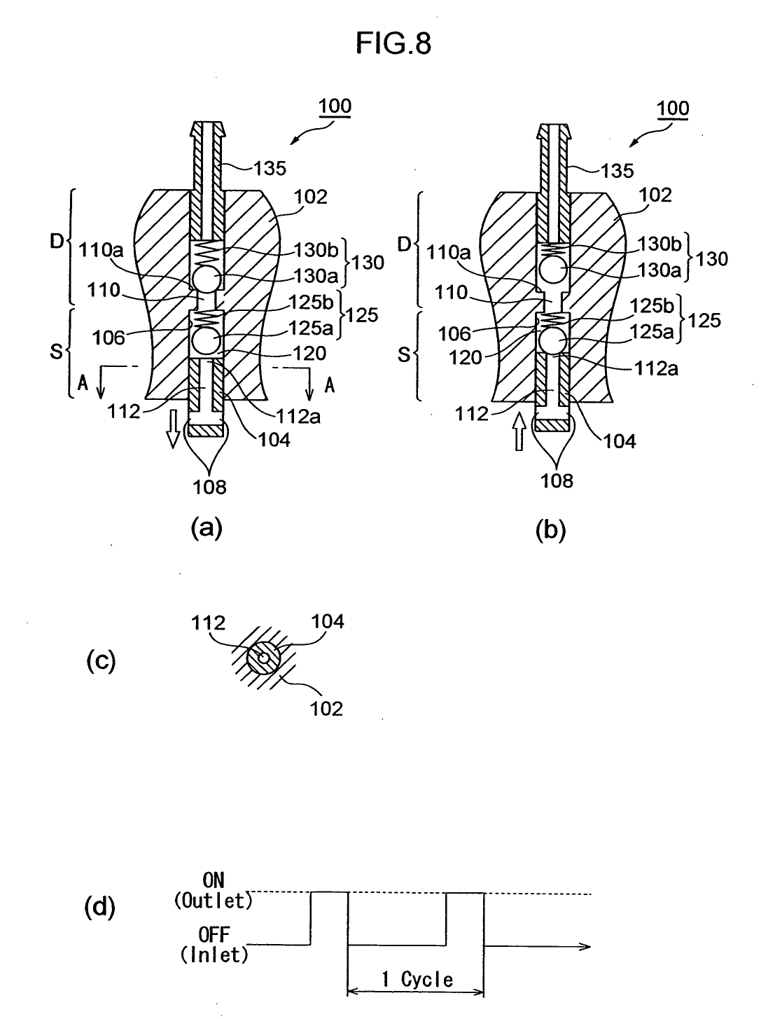

In addition, for a period during which the plunger 4 is thus held at the top dead center position of its stroke, the pump chamber 20 that is a pump chamber is kept at a sealed state (because the opening 19 of the fluid suction passage 12 is closed by the plunger 4 and the inlet 8 and outlet 10 are not communicated with each other basically), and a clearance seal is provided between the plunger 4 and cylinder 2, whereby it is possible to suppress the so-called blow-by phenomenon where the fluid from a fluid source is sucked from the inlet 8 and leaks from the outlet 10 when the pressure on the discharge side D is lower than the pressure on the suction side S. In contrast thereto, in the conventional structure as shown in FIG. 8, if the plunger 104 is held at the top dead center position of its stroke, there is a possibility that the suction valve 125 is opened by a pressure difference between the suction side S and discharge side D, and it is thus difficult to completely suppress the blow-by phenomenon.

After the plunger 4 is held at the top dead center position of its stroke for a predetermined time as described above, the application of current to the electromagnetic coil is halted (OFF state) at predetermined timing, and the plunger 4 is pulled back again to the bottom dead center of its stroke by the escape operation of the driving core of the solenoid. Then, when the opening 19 of the fluid suction passage 12 is opened by the plunger 4, the pump chamber 20 is communicated with the inlet 8, and the operation shifts to the suction operation as described previously. Then, such a series of suction/discharge operation is carried out repeatedly with ON/OFF of the application of current to the electromagnetic coil as one cycle, as shown in FIG. 1(d). In addition, as means for achieving such voltage control, there may be a mechanical adjustment of cam timing and program control of electrically controllable actuator other than the solenoid.

As described above, in the plunger pump 1 of this embodiment, since the opening 19 of the fluid suction passage 12 opened to the pump chamber 20 is opened and closed by the plunger 4 itself, in other words, the plunger 4 is provided with the valve function (the plunger 4 serves as a suction valve on the suction side S), the need is eliminated of providing a valve (for example, the suction valve 125 as shown in FIG. 8) on the suction side S. Therefore, as compared with the conventional case, the number of components is decreased, the valve structure is simplified, the assembly is also simplified, and it is possible to miniaturize the entire plunger pump 1.

Further, since any members such as a valve are not present inside the pump chamber 20, the issue of the dead volume in the pump chamber 20 is also resolved, and it is possible to use the entire inner capacity of the pump chamber 20 effectively. The compression rate (pump performance) is thus improved, and it is possible to achieve a desired structure in increases in discharge pressure and air exclusion.

Furthermore, since any members are not present (such as, for example, the valve body 125a of the suction valve 125 as shown in FIG. 8) that collide with the plunger 4 with opening and closing of the opening 19 of the fluid suction passage 12, it is possible to minimize the vibration and noise at the time the pump is operating, and to avoid wear of the valve structure and occurrence of failure in sealing inside the pump chamber 20 due to the wear.

Moreover, in the plunger pump 1 of this embodiment, since a discharge amount of fluid discharged from the outlet 10 is specified by the top dead center of the stroke of the plunger 4 and a position of the opening 19 of the fluid suction passage 12, it is possible to change a discharge amount (obtain a required discharge flow rate) with ease only by merely changing a position of the opening 19 (accordingly, with simplified processing) without changing the stroke of the plunger 4 and inner diameter of the continuous hole 6. In other words, only by changing a position of the opening 19, it is possible to vary a ratio of a suction stroke and discharge stroke to the entire stroke of the plunger 4.

Further, in the plunger pump 1 of this embodiment, since the fluid suction passage 12 is formed on the inner surface of the continuous hole 6 along the axis direction thereof, the extending direction of the fluid suction passage 12 is in accordance with the extending direction of the continuous hole 6, and it is thereby possible to perform processing on the fluid suction passage 12 in the same direction as in processing on the continuous hole 6.

Furthermore, in the plunger pump 1 of this embodiment, since the plunger 4 is held at the top dead center position of its stroke for a predetermined time, it is possible to minimize the time the plunger 4 is held at the bottom dead center position of its stroke i.e. at the position having the risk of occurrence of the blow-by phenomenon in one cycle of suction/discharge operation. In other words, it is possible to reduce to a minimum the time percentage of occurrence of the blow-by phenomenon by a pressure difference between the suction side S and discharge side D, and to secure a proper discharge amount.

Moreover, in this embodiment, since the voltage V₂ applied to the electromagnetic coil to keep the plunger 4 at the top dead center position for a predetermined time is set lower than the application voltage V₁ required to slide the plunger 4 to the top dead center position, power savings can be achieved (power consumption can be reduced).

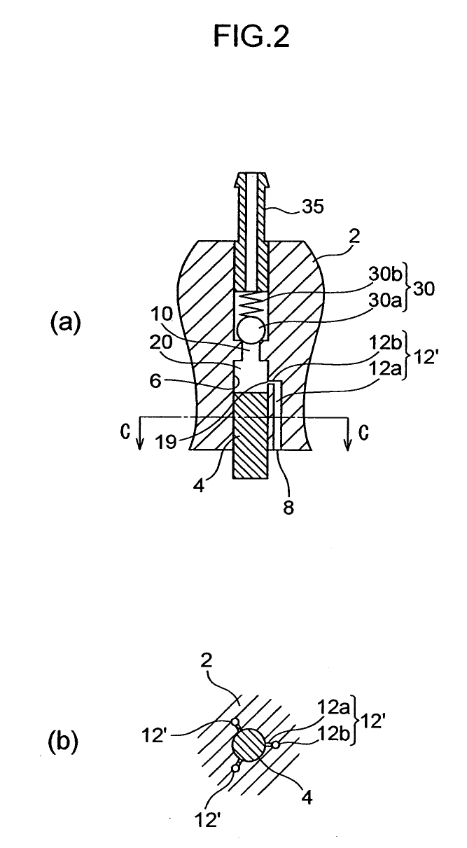

In addition, as the fluid sucked and discharged by the plunger pump 1 of this embodiment, various types of fluids are considered such as oils including lubricant oil and gasoline. In this embodiment, the size and number of the fluid suction passages 12 can be set optionally corresponding to a required discharge amount. Further, the fluid suction passage 12 is formed on the inner surface of the continuous hole 6 along the axis direction thereof in this embodiment, but may be formed independently of the continuous hole 6 as shown in FIG. 2. In other words, each of fluid suction passages 12′ as shown in FIG. 2 is comprised of a first passage 12a which is formed in the cylinder 2 independently of the continuous hole 6 and extends in parallel with the continuous hole 6, and a second passage 12b which extends in the direction perpendicular to the first passage 12a and serves as a horizontal hole to cause the first passage 12a to communicate with the pump chamber 20.

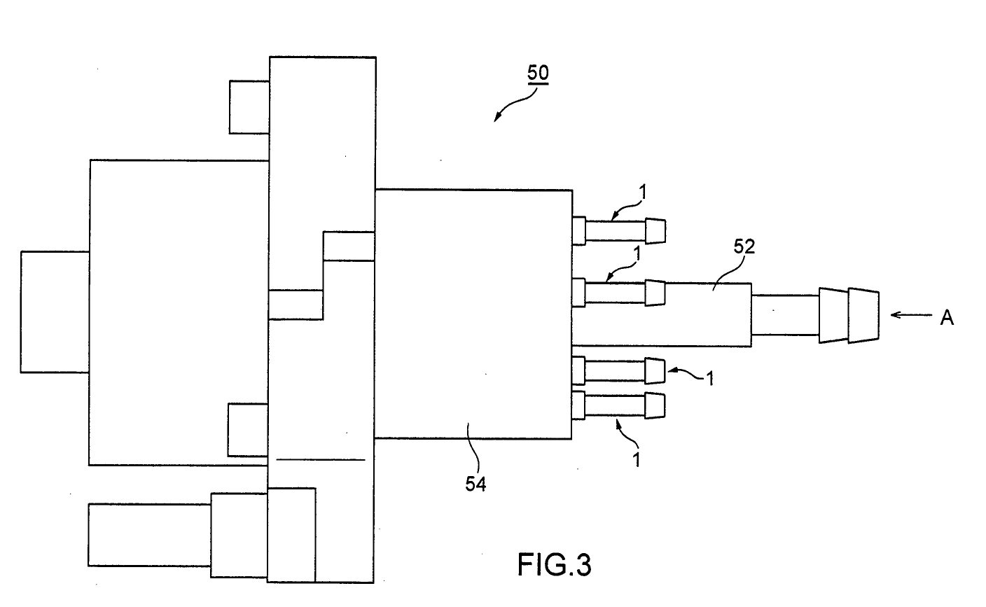

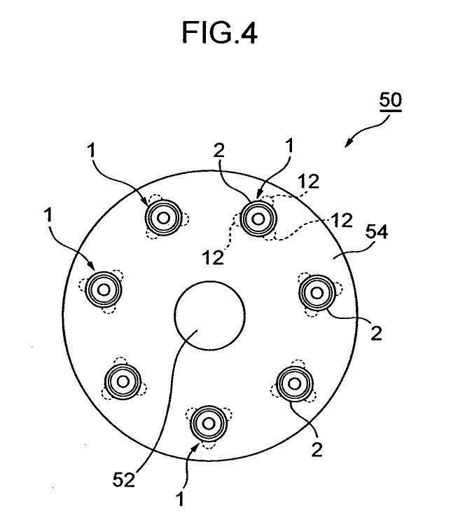

FIGS. 3 to 5 show a multi-discharge type plunger pump 50 provided with a plurality of plunger pumps 1 as shown in FIG. 1. The multi-discharge type plunger pump 50 is configured, for example, as a seven-discharge electromagnetic oil pump for two-cycle engine division refueling provided with seven plunger pumps 1 (accordingly, having seven outlets), and has a common suction duct 52 communicating with an oil source (fluid source) not shown. The cylinder (cylinder part) 2 of each of the plunger pumps 1 is formed integrally with a housing 54 of the multi-discharge type plunger pump 50. In addition, the structure and operation of each of the plunger pumps 1 is already explained in FIG. 1, and therefore, the plunger pumps are assigned the same reference numerals as in FIG. 1 to omit specific descriptions thereof.

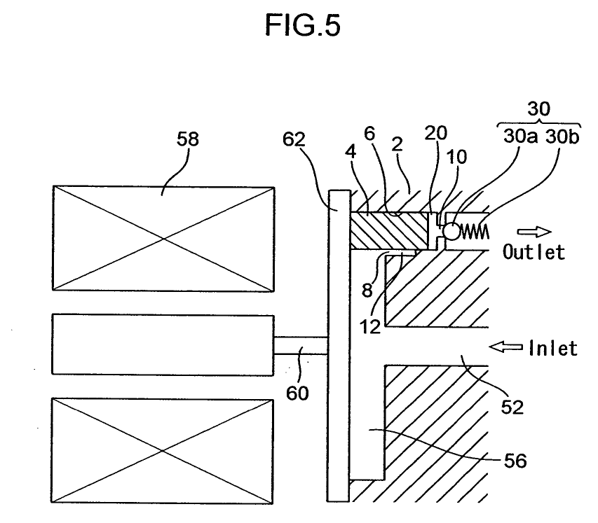

As is shown distinctly in FIG. 5, the suction duct 52 is communicated with a suction pump chamber 56 shared by the plunger pumps 1, and the inlet 8 of each of the plunger pumps 1 is opened to the suction pump chamber 56. The plunger 4 of each of the plunger pumps 1 is attached to a common driving plunger 62 that slides all the plungers at the same time in conjunction with one another, and reciprocates by the driving plunger 62 going back and forth due to the operation of a solenoid driving core 60 with the application of voltage to an electromagnetic coil 58 constituting a solenoid.

In this embodiment, a fluid sucked from one side of the multi-discharge type plunger pump 50 via the suction duct 52 enters the pump chamber 20 from the fluid suction passages 12 of each of the plunger pumps 1 while being inverted via the suction pump chamber 56, and is discharged from the one side where the suction duct 52 is situated in the opposite direction to the suction direction.

Further, in this embodiment, at least some of seven outlets 10 are different from one another in discharge amount of the fluid to discharge. More specifically, the position of the opening 19 of the fluid suction passage 12 differs among the plunger pumps 1.

Thus, by using the plunger pump 1 with the structure as shown in FIG. 1, as in this embodiment, even in the case of operating a plurality of plungers 4 by a common driving part and supplying different flow amounts from the outlets 10 at a constant discharge pitch with the same stroke set on all the plungers 4, it is only required to change a position of the opening 19 of each of the fluid suction passages 12 without changing an inner diameter of the continuous hole 6 for each of the plungers 4, and the processing is thus easy. In other words, in a multi-discharge structure that operates a plurality of plungers 4 in conjunction with one another, it is possible to change a setting of flow rate in each pump chamber 20 only by changing a position of the opening 19 of the corresponding fluid suction passage 12, and variations as a pump can thus be dramatically extended.

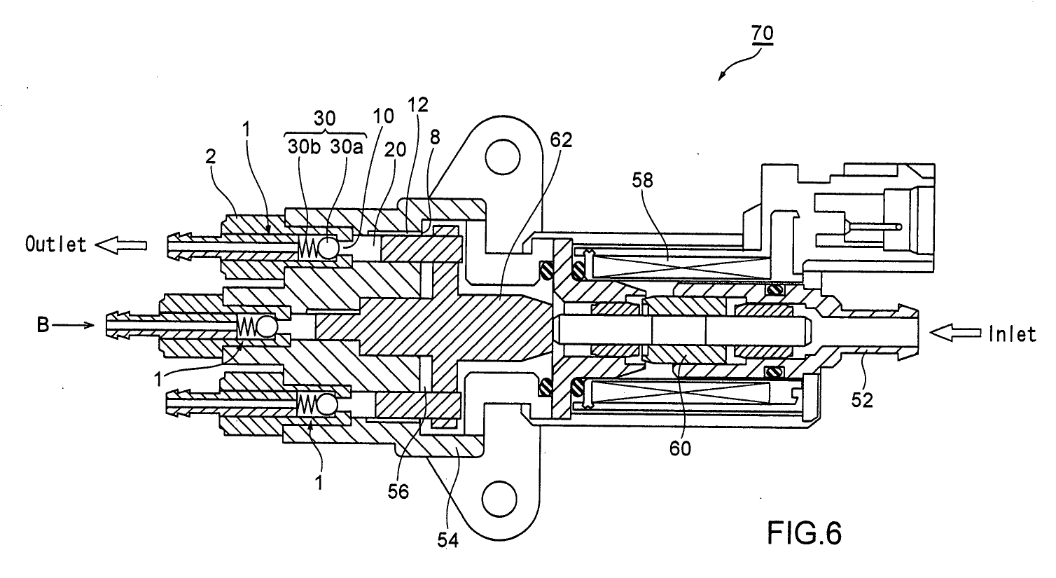

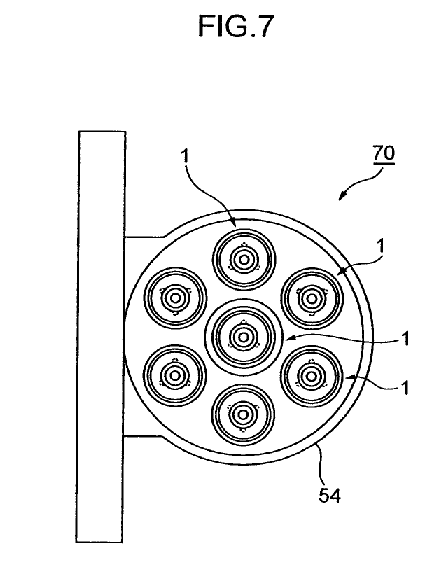

FIGS. 6 and 7 show another embodiment of the multi-discharge type plunger pump, for example, used in an outboard motor. The multi-discharge type plunger pump 70 has the same basic structure as in the multi-discharge type plunger pump 50 as shown in FIGS. 3 to 5, and is assigned the same reference numerals to omit specific descriptions thereof, but different from the multi-discharge type plunger pump 50 in the fluid suction/discharge direction that is linear. In other words, the fluid sucked from one side of the multi-discharge type plunger pump 70 via the suction duct 52 is flowed linearly via the suction pump chamber 56, enters the pump chamber 20 from the fluid suction passages 12 of each of the plunger pumps 1, and is discharged from the other side opposed to the side where the suction duct 52 is situated in the same direction as the suction direction.

In addition, the present invention is not limited to the above-mentioned embodiments, and is capable of being carried into practice with various modifications thereof. For example, in each of the above-mentioned embodiments, each plunger 4 is provided with a single outlet 10, but may be provided with a plurality of outlets 10. In this case, each of the outlets 10 is naturally provided with the discharge valve 30.

The present invention relates to a plunger pump that sucks a constant amount of fluid from a fluid source to discharge and to a method of controlling discharge of the pump, and thus has the industrial applicability. The plunger pump is applicable to various plunger pumps that suck a variety of fluids to discharge.

1. A plunger pump comprising:
a cylinder having an inlet to suck a fluid from a fluid source and an outlet to discharge the sucked fluid;
a continuous hole which is formed inside the cylinder and communicated with the outlet;
a plunger which is inserted in the continuous hole to be slidable and forms a pump chamber to suck and discharge the fluid with the outlet, the pump chamber being formed between the plunger and the outlet; and
a fluid suction passage which is formed in the cylinder or the plunger, to suck a fluid into the pump chamber,
wherein an opening of the fluid suction passage opened to the pump chamber is opened and closed by the plunger sliding inside the continuous hole.

2. The plunger pump according to claim 1, further comprising:
a valve body which is provided to close the outlet, and opens the outlet only when receiving a predetermined pressure generated inside the pump chamber by the plunger sliding toward the outlet.

3. The plunger pump according to claim 1, wherein a discharge amount of the fluid discharged from the outlet is determined by a top dead center of a stroke of the plunger and a position of the opening of the fluid suction passage.

4. The plunger pump according to any one of claims 1 to 3, wherein the fluid suction passage is formed on the inner surface of the continuous hole along the axis direction thereof.

5. The plunger pump according to claim 1, further comprising:
a driving part that slides the plunger inside the continuous hole.

6. The plunger pump according to claim 5, wherein the driving part slides the plunger inside the continuous hole by electromagnetic activation force generated by applying current to an electromagnetic coil.

7. The plunger pump according to claim 5, wherein the driving part holds the plunger at a top dead center position of a stroke of the plunger for a predetermined time.

8. The plunger pump according to claim 7, wherein after the plunger reaches the top dead center position of the stroke, the driving part holds the plunger at the top dead center position for a predetermined time by maintaining a voltage lower than an application voltage to the electromagnetic coil required to slide the plunger to the top dead center position.

9. The plunger pump according to claim 1, wherein the fluid is oils including lubricant oils or gasoline.

10. A multi-discharge type plunger pump comprising:
a suction duct to suck a fluid from a fluid source;
a plurality of cylinder parts each having an inlet communicated with the suction duct and an outlet to discharge the sucked fluid;
continuous holes each of which is formed inside respective one of the cylinder parts and communicated with the outlet;
plungers each of which is inserted in respective one of the continuous holes to be slidable and forms a pump chamber to suck and discharge the fluid with the outlet, the pump chamber being formed between each of the plungers and the outlet; and
a plurality of fluid suction passages each of which is formed in respective one of the cylinder parts or the plungers inserted in respective one of the continuous holes, to suck a fluid into the pump chamber,
wherein an opening of each of the fluid suction passages opened to the pump chamber is opened and closed by respective one of the plungers sliding in respective one of the continuous holes.

11. The multi-discharge type plunger pump according to claim 10, further comprising:
valve bodies each of which is provided to close the outlet, and opens the outlet only when receiving a predetermined pressure generated inside the pump chamber by respective one of the plungers sliding toward the outlet.

12. The multi-discharge type plunger pump according to claim 11, wherein a discharge amount of the fluid discharged from the outlet is determined by a top dead center of a stroke of each of the plungers and a position of the opening of respective one of the fluid suction passages.

13. The multi-discharge type plunger pump according to claim 12, wherein at least some of outlets are different from one another in the discharge amount of discharged fluid.

14. The multi-discharge type plunger pump according to claim 11, wherein each of the fluid suction passages is formed on the inner surface of respective one of the continuous holes along the axis direction thereof.

15. The multi-discharge type plunger pump according to claim 11, further comprising:
a driving part that slides the plungers respectively inside the continuous holes.

16. The multi-discharge type plunger pump according to claim 15, wherein the driving part slides all the plungers at the same time in conjunction with one another.

17. The multi-discharge type plunger pump according to claim 15,
wherein the driving part slides the plungers respectively inside the continuous holes by electromagnetic activation force generated by applying current to an electromagnetic coil.

18. The multi-discharge type plunger pump according to claim 15,
wherein the driving part holds each of the plungers at a top dead center position of a stroke thereof for a predetermined time.

19. The multi-discharge type plunger pump according to claim 18,
wherein after each of the plungers reaches the top dead center position of the stroke, the driving part holds the each of the plungers at the top dead center position for a predetermined time by maintaining a voltage lower than an application voltage to the electromagnetic coil required to slide the each of the plungers to the top dead center position.

20. The multi-discharge type plunger pump according to claim 10,
wherein the fluid is oils including lubricant oils or gasoline.

21. A method of controlling discharge of a plunger pump comprising a cylinder having an inlet to suck a fluid from a fluid source and an outlet to discharge the sucked fluid, a continuous hole which is formed inside the cylinder and communicated with the outlet, a plunger which is inserted in the continuous hole to be slidable and forms a pump chamber to suck and discharge the fluid with the outlet, the pump chamber being formed between the plunger and the outlet, a valve body which is provided to close the outlet and opens the outlet only when receiving a predetermined pressure generated inside the pump chamber by the plunger sliding toward the outlet, and a fluid suction passage which is formed in the cylinder or the plunger, to suck a fluid into the pump chamber, in which an opening of the fluid suction passage opened to the pump chamber is opened and closed by the plunger sliding inside the continuous hole,
wherein after reaching a top dead center position of a stroke of the plunger, the plunger is held at the top dead center position for a predetermined time.

22. The method of controlling discharge according to claim 21, wherein the plunger is slid inside the continuous hole by electromagnetic activation force generated by applying current to an electromagnetic coil, and is held at the top dead center position for a predetermined time by maintaining a voltage lower than an application voltage to the electromagnetic coil required to slide the plunger to the top dead center position, after reaching the top dead center position of the stroke.

23. The multi-discharge type plunger pump according to claim 12, wherein each of the fluid suction passages is formed on the inner surface of respective one of the continuous holes along the axis direction thereof.

24. The multi-discharge type plunger pump according to claim 13, wherein each of the fluid suction passages is formed on the inner surface of respective one of the continuous holes along the axis direction thereof.
